(19) United States
(12) Patent Application Publication
(10) Pub. No.: US 2006/0122459 A1
(43) Pub. Date: Jun. 8, 2006

(54) Removable resectoscope provided with an external shaft

(75) Inventor: Thomas Aue, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, HAMBURG (DE)

(21) Appl. No.: 10/525,257

(22) PCT Filed: Aug. 6, 2003
(86) PCT No.: PCT/EP03/08760

§ 371 Date: Feb. 22, 2005

(30) Foreign Application Priority Data
Sep. 12, 2002 (DE) 102 42 608.2

(51) Int. Cl. A61B 1/00

(52) U.S. Cl. 600/105

(57) ABSTRACT

A urological resectoscope (1) including a main body (2) that is traversed by an optics (9) and to which the guide (4) of a carriage (6) is affixed proximally and to which are mounted an inner shaft (15) and an outer shaft (16). The inner and outer shaft both extend distally from the main body. The outer shaft (16) encloses the inner shaft (15) and is detachable from the main body via an externally actuated outer connector element (17). The outer connector element (17) is connected to the main body (2).

### BACKGROUND OF THE INVENTION

For decades already resectoscopes have been the backbone of urological instrumentation. For permanent rinsing they comprise an inner and an outer shaft, rinsing fluid being fed from the inner shaft and then being evacuated through the annular space between said shafts. The outer shaft is detachable by means of an externally actuated connector element. The optics monitoring the surgery zone, and the implement operational therein, for instance the conventional high-frequency (hf) cutting loop, run through the inner shaft.

Regarding known resectoscopes of the species described in U.S. Pat. Nos. 5,807,240 and 5,486,155, the inner shaft also is detachable by means of an externally actuated connector element. The connector elements for the outer and inner shafts hereafter are called external connector element and inner connector element, respectively. In known resectoscopes, the inner connector element also is actuated externally and it directly engages the resectoscope main body, whereas the outer connector element engages the inner connector element. It is furthermore known to make the outer connector element rotatable relative to the entire remaining resectoscope, including the inner shaft.

One of the main problems encountered in resectoscopes is the available length of the shaft that can be inserted into the typical human body. The length should be as large as possible whereas the total resectoscope length should be minimized in order to minimize, for instance, the optics which extend the length of the resectoscope and for optical reasons should be as short as possible. Accordingly, designers in this field attempt foremost to maximize the shaft length while reducing resectoscope length elsewhere, but difficulties arise.

The design of known resectoscopes comprising two externally actuated connector elements, inherently limits the available shaft length.

### BRIEF SUMMARY OF THE INVENTION

The objective of the present invention is to create a resectoscope of simple design offering greater available shaft length.

In the present invention, the outer connector element directly engages the resectoscope main body. On the other hand no external connector element is used to actuate the inner connector element and as a result, contrary to the case of conventional design, the length of one externally driven connector element, namely a minimum of about 5 mm, may be saved and, keeping the total resectoscope length constant, the available outer shaft length may be increased. This feature is of substantial advantage to the surgeon. The outer connector element directly engages the resectoscope main body. In this manner, that is circumventing external action on parts of an inner connector element placed at the resectoscope periphery, the connection zone may be shortened. The inner shaft in this design is affixed within the outer connector element.

The inner shaft may be affixed to the resectoscope main body, preferably in a permanent manner. This very simple design entirely circumvents the known inner connector element.

By providing an inner connector element to remove the inner shaft, conventional instrument cleansing can be improved. The inner connector element may exhibit a variety of designs, for instance being a screw connection, bayonet connection, snap-in connection with elastic tongues or the like. The inner connector element need not exert substantial retaining forces because during use the inner shaft is protected by the outer shaft against mechanical stresses.

Illustratively, the inner shaft may be of constant cross-sectional shape and smooth as far as its proximal end by which it may be inserted into a resectoscope main body borehole where it might be permanently soldered or welded into place. The inner shaft also may be connected by means of a thread or a bayonet lock to the borehole, or it may be merely plugged into the borehole and remain therein in a frictional or press-fit manner. Such a design is radially compact and consequently the enclosing outer connector also may be made compact and the entire assembly slender.

Thereby, the inner shaft is affixed not to the resectoscope main body but to the outer shaft, namely to this outer shaft's proximal end zone. Illustratively, the inner shaft may be inserted by its widened proximal end zone into the proximal end zone of the outer shaft and soldered to it.

The inner shaft also may be detachably affixed to the outer shaft, similarly to the way it may be detachably fastened to a borehole in the resectoscope main body.

Thereby, the inner shaft is fitted with a connector element that, upon closure of the outer connector element, shall engage between the outer shaft and the resectoscope main body and in this way can be clamped into place when the outer connector element is closed. This feature offers a simple design variant where, however, the inner shaft can be removed only in the proximal direction from the outer shaft after the outer shaft has been detached from the main body—whereas, in other designs, the inner shaft may be removed in the distal direction from the outer shaft after loosening the inner connector element.

Independently of the design of the invention of the inner shaft affixation, the outer connector element may be conventionally rotatable. Even regarding the configuration of the feed and drain ducts at the outward hookup stubs, known designs may be used, including a rotatable outer connector element.

The invention is shown illustratively and schematically in the appended drawings.

### DETAILED DESCRIPTION OF THE INVENTION

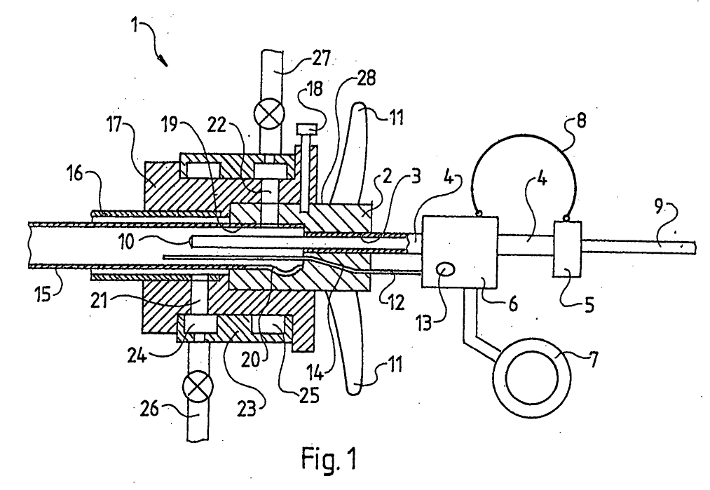

FIG. 1 is a section of a first embodiment of a resectoscope of the invention, and

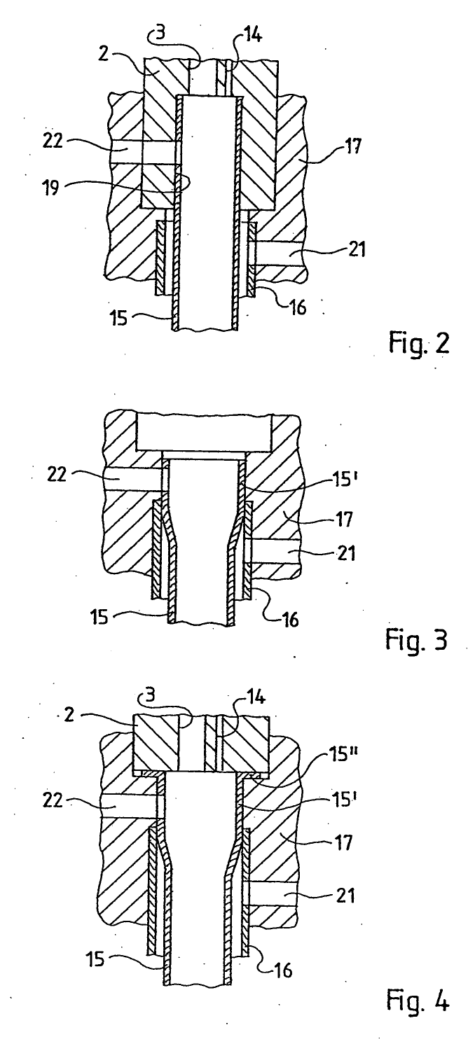

FIGS. 2-4 show three further embodiments of details of FIG. 1.

### DETAILED DESCRIPTION OF THE INVENTION

The resectoscope 1 shown in FIG. 1 comprises a main body 2 to which an optics guide tube 4 is affixed inside a borehole 3, the tube 4 passing proximally a distance from the main body 2 through an optics guide plate 5 within which it is affixed.

A carriage 6 with a thumb ring 7 runs on the optics guide tube 4 and is connected by means of a leaf spring 8 with the optics guide tube 5. As shown by FIG. 1, an optics 9 having an objective 10 can be inserted by means of the optics guide tube 4 in the distal direction, far beyond the main body 2.

By holding the implement in one hand, the surgeon may actuate finger grips 11 at the main body 2 to displace the carriage 6 in the axial direction of the resectoscope 1 to move an elongated support 12 affixed to the carriage 6, indicated at 13, through a duct 14 in the main body 2 far distally beyond the main body in order to reciprocate a surgical instrument (omitted in FIG. 1), for instance an hf-loaded cutting loop, which is mounted at the end of the support 12.

An inner shaft 15 enclosing the optics 9 and the support 12 is affixed to the main body 2. An outer shaft 16 enclosing the inner shaft 15 also is affixed to the main body 2. The shafts 15, 16 illustratively are cross-sectionally circular and configured mutually coaxially.

The tubular outer shaft 16 is affixed at its proximal end to an outer connector element 17 which, as shown, encloses the main body 2 and is detachably affixed by a locking pin 18 or some other means to the main body. The main body 2 supporting the outer connector element 17 comprises a borehole 19 within the outer surface 28 of the main body, the borehole 19 receiving the proximal terminal zone of the inner shaft 15. The inner shaft 15 is fitted in its proximal terminal zone and at one site of its circumference with a resilient lip 20 engaging in a securing manner a matching radial clearance in the borehole 19, as a result of which once inserted into the borehole 19, the inner shaft 15 is elastically secured, though, the inner shaft 15 can be retracted again when the spring force is overcome.

The gap between the shafts 15, 16 may communicate with the outside through a radially configured borehole 21 passing through the outer connector element 17 and the outer shaft 16 firmly affixed to it. The inside space of the inner shaft 15 may communicate with the outside through a borehole 22 passing through the inner shaft 15, the main body 2 and the outer connector element 17.

In this embodiment, a hookup ring 23 rests rotatably on the outer surface of the outer connection element 17 and is fitted with circumferential ducts 24 and 25 in the axial position of the boreholes 21 and 22, the ducts each communicating through valve-controlled hookup stubs 26, 27 to the outside in order to be connected as needed to evacuation or rinsing hoses.

In the illustrated embodiment, the outer shaft 16, jointly with the outer connector element 17, may be removed from the main body 2 following withdrawal of the locking pin 18. In the process the inner shaft 15 may remain at the main body 2 and then be pulled out of the borehole 19. This design furthermore allows seizing only the inner shaft 15 at the distal end and to remove it first through the outer shaft 16 that is still in place.

FIGS. 2 through 4 show three alternative embodiments as a segment of the central region of FIG. 1. As far as feasible these FIGS. 2-4 retain the design details of FIG. 1 and also their references.

FIG. 2 shows an embodiment wherein the inner shaft 15 is a smooth tube—in particular devoid of the elastic lip 20—inserted into the borehole 19 of the main body 2. The inner shaft 15 may be affixed inside the borehole 19 for instance by soldering. In this embodiment, the inner shaft 15 therefore is rigidly affixed to the main body 2 whereas the outer shaft 16 is detachable as in FIG. 1 on account of the outer connector element 17.

However, the outer shaft 15 may also be detachably connected to the main body 2 in the manner shown in FIG. 2. Illustratively, the inner shaft 15 and the borehole 19 may be threaded, as a result of which the inner shaft 15 can be screwed into the main body 2.

Moreover, the borehole 19 may be eliminated. In that case, the inner shaft 15 may be affixed in another way to the distal end face of the main body 2 which, then however must, at a minimum, allow the optics 9 and the support 12 to move into the inner shaft 15.

FIG. 3 shows another embodiment wherein the outer shaft 16 is affixed to the outer connector element 17 exactly as in the embodiment of FIG. 1 and can be connected by the element 17 to the main body 2 which is omitted from FIG. 3. In this embodiment of FIG. 3, the inner shaft 15 is widened in its terminal zone 15′ to be the size of the inside diameter of the outer shaft 16 or of the outer connector element 17, and is inserted into the outer shaft 16 and/or connector element 17, as shown in FIG. 3. The inner shaft 15 may be clamped in position in this configuration, or illustratively it may be rigidly joined for instance by soldering to the outer shaft 16 respectively the main body 17.

The design of FIG. 3 also allows connection of the inner shaft 15 in a detachable manner. Illustratively, it may be fitted in its widened distal terminal zone 15′ with an elastic lip 20 as shown in FIG. 1, the lip elastically engaging a corresponding clearance in the outer connector element 17. Bayonet and screw connections also are applicable at this site.

Another embodiment is shown in FIG. 4. The inner shaft 15 again is widened in its proximal terminal zone 15′ but—unlike the embodiment of FIG. 3—it comprises at its proximal end an outer flange 15″ which engages between the outer connector element 17 and the main body 2 when these are affixed to each other and which, once the outer connector element 17 has been locked, this flange 15″ shall be fixed in place. After the connection is dissolved and the outer connector element 17 is removed from the main body 2, the inner shaft 15 may be pulled out of the outer shaft 16 in the proximal direction.

The outer connector element 17 also may be designed in a manner other than shown in FIGS. 1 through 4, for instance, the rotatable hookup stub 23 may be omitted and the hookup stubs 26, 27 may be applied directly against the boreholes 21, 22. Moreover, the outer connector element 17 may be supported in a rotatable manner on the main body 2. In that case, and keeping the locking pin 18, this pin might run in an outer groove of the main body 2. In this embodiment, the annular ducts 24, 25 might run through the boreholes 21, 22 on the inside of the outer connector element 17.

If, in the above embodiment, the inner shaft 15 is permanently affixed to the main body, assembly will require inserting the implement support 12 from the distal side—contrary to conventional assembly.

1. A urological resectoscope (1) comprising a main body (2) traversed by an optics (9), a guide (4) of a slide carriage (6) being proximally affixed to said main body, an inner shaft (15), and an outer shaft (16), said outer shaft, which encloses said inner shaft and which is detachable by an externally actuated outer connector element (17), being mounted on said main body (2) and extending distally therefrom, wherein the outer connector element (17) is connected to the main body (2).

2. The resectoscope as claimed in claim 1, wherein the inner shaft (15) is affixed to the main body (2).

3. The resectoscope as claimed in claim 2, wherein the inner shaft (15) is detachably affixed to the main body (2) by an inner connector element (20).

4. The resectoscope as claimed in claim 2, wherein the inner shaft (15) is affixed in a borehole (19) of the main body (2).

5. The resectoscope as claimed in claim 1, wherein the inner shaft (15) is affixed to a proximal terminal zone of the outer shaft (16).

6. The resectoscope as claimed in claim 5, wherein the inner shaft is detachably affixed by an inner connector element (20) to a proximal terminal zone of the outer shaft (16).

7. The resectoscope as claimed in claim 1, wherein the inner shaft (15) comprises a connector element (15″) engaging the outer connector element (17) between the outer shaft (16, 17) and the main body (2).
